(19) United States
(12) Patent Application Publication
(10) Pub. No.: US 2024/0408237 A1
(43) Pub. Date: Dec. 12, 2024

(54) METHOD AND PHARMACEUTICAL COMPOSITION FOR TREATING A CARTILAGE DAMAGE USING SOX9 GENE

(71) Applicants: FAR EASTERN MEMORIAL HOSPITAL, New Taipei City (TW); NATIONAL YANG MING CHIAO TUNG UNIVERSITY, Taipei City (TW)

(72) Inventors: Hsiu-Jung LIAO, New Taipei City (TW); Chih-Hung Chang, New Taipei City (TW); Chi-Ying Huang, Taipei City (TW); Ly James Lee, Taipei City (TW); Tai-Shan Cheng, Taipei City (TW); Sin-Yu Chen, Taipei City (TW)

(73) Assignees: FAR EASTERN MEMORIAL HOSPITAL, New Taipei City (TW); NATIONAL YANG MING CHIAO TUNG UNIVERSITY, Taipei City (TW)

(21) Appl. No.: 18/737,607

(22) Filed: Jun. 7, 2024

Related U.S. Application Data
(60) Provisional application No. 63/506,917, filed on Jun. 8, 2023.

(51) Int. Cl. A61K 48/00; A61K 9/00; A61K 9/50; A61K 38/39; A61K 45/06; A61P 19/02; C12N 15/88

(52) U.S. Cl. CPC A61K 48/005; A61K 9/0019; A61K 9/5063; A61K 38/39; A61K 45/06; A61K 48/0075; A61K 48/0083; A61P 19/02; C12N 15/88

(57) ABSTRACT

The present invention is related to a method and pharmaceutical composition for treating a cartilage damage in a subject (including a human or an animal), particularly osteoarthritis (OA), using extracellular vesicles (EVs) with SOX9 gene, called as “EV-SOX9”. The EV-SOX9 is obtained by encapsulating the SOX9 mRNA or the mRNA of its upstream and downstream gene in EVs, naïve EVs with high expression level of SOX9 mRNA or its upstream and downstream gene from different cell sources, or MSC-derived EV-SOX9, which is obtained by transferring the SOX9 gene or its upstream and downstream gene into a multipotent cell and collecting EVs.

### FILED OF THE INVENTION

This non-provisional application claims the benefit under 35 U.S.C. § 119 (e) to U.S. Provisional Application No. 63/506,917, filed on Jun. 8, 2023, all of which are hereby expressly incorporated by reference into the present application.

The invention concerns a method and pharmaceutical composition for therapy of a cartilage damage.

### BACKGROUND

Cartilage is a tissue composed of extracellular matrix and cartilage cells as chondrocyte, which has neither no blood vessels nor nerve. Therefore, it is difficult to heal the cartilage tissue when it is damaged. Further, chondrocytes are surrounded by hard extracellular matrix, and thus it is difficult to regenerate cartilage once damaged or degenerated.

For treatments of a cartilage damage or degeneration, some drugs such as analgesics, steroids, or non-steroidal anti-inflammatory drugs may be used clinically. However, the drug treatment only has an effect of relieving the pain or the inflammatory reaction itself non-specifically. Cartilage protectants such as hyaluronic acid, glucosamine, or chondroitin may also be used to treat damaged cartilage tissue. However, the cartilage protectants temporarily protect the joints by merely nourishing the cartilage cells or alleviating the shocks. In addition, surgical procedures such as arthroscopic surgery, proximal tibial osteotomy, total knee arthroplasty, or bone-cartilage tissue grafting may be used. Various orthopedic surgical procedures are performed, and representative methods therefor include bone marrow stimulation and osteochondral graft. The bone marrow stimulation is an alternative method with stem cells, for example those derived from bone marrow, but it is regenerated as fibrocartilage rather than hyaline cartilage after surgery. The osteochondral graft is a treatment method by collecting osteo-cartilage connective tissue at a site that receives less weight in the patient's own cartilage tissue and then transplanting it to the cartilage damage site. However, it cannot be used for a large damage site. In addition, these therapies cannot completely prevent progressive joint destruction and cure degenerative osteoarthritis (OA).

Therefore, it is still derivable to develop a new method and composition for treating a cartilage damage, particularly osteoarthritis (OA).

### SUMMARY OF THE INVENTION

Accordingly, the present invention provides a novel approach for treating a cartilage damage, particularly osteoarthritis (OA), using SOX9 mRNA in extracellular vesicles (EVs), called as “EV-SOX9” hereinafter.

In one aspect, the present invention provides a method for treating a cartilage damage in a subject comprising the steps of:
providing an EV-SOX9, which is extracellular vesicles (EVs) with SOX9 gene or its upstream or downstream gene;
administrating to the subject a therapeutically effective amount of the EV-SOX9.

In another aspect, the present invention provides a pharmaceutical composition for treating a cartilage damage, comprising a therapeutically effective amount of EV-SOX9, and a pharmaceutically acceptable carrier.

In a further aspect, the present invention provides a use of EV-SOX9 in preparing a therapeutic agent for treating a cartilage damage.

In some embodiments of the invention, the SOX9 gene may be its upstream, or downstream gene, such as Collagen type II (COL2A1) or Aggrecan (ACAN).

In one embodiment of the invention, the EV-SOX9 is obtained by encapsulating the SOX9 mRNA, the mRNA of its upstream or downstream gene, or an miRNA, which regulates SOX9 in extracellular vesicles (EVs).

In another embodiment of the invention, the EV-SOX9 is naïve EVs with high expression level of SOX9 mRNA or its upstream or downstream gene from different cell sources.

In a further embodiment of the invention, the EV-SOX9 is mesenchymal stem cells (MSCs)-derived EV-SOX9, which is obtained by transferring the SOX9 gene or its upstream or downstream gene, such as SOX9 mRNA, into mammalian cells and collecting extracellular vesicles (EVs) derived from the mammalian cells.

In some embodiments of the invention, the mammalian cells comprise multipotent cells such as mesenchymal stem cells (MSCs), or fibroblasts such as murine embryonic fibroblasts (MEFs), or 293T cells.

In one preferred example of the invention, the multipotent cells are mesenchymal stem cells (MSCs).

In one embodiment of the invention, the EV-SOX9 is administered to the cartilage damage of the subject via an injection or a surgery.

In a further aspect, the method for treating a cartilage damage according to the invention further comprises a combination of a therapy or a therapeutic agent for treating a cartilage damage, such as platelet-rich plasma (PRP), hyaluronic acid (HA), a cell therapy for repair or regeneration of cartilage tissues such as autologous chondrocyte Implantation (ACI), and/or a stem cell therapy.

In one particular example of the present invention, the cartilage damage is osteoarthritis (OA).

In one particular embodiment of the invention, the method for treating a cartilage damage in a subject comprises the steps of:
providing MSC-derived EV-SOX9, which is obtained by transferring the SOX9 gene or its upstream or downstream gene into mesenchymal stem cells (MSCs) and collecting extracellular vesicles (EVs) derived from the MSCs as MSC-derived EV-SOX9;
administrating to the subject a therapeutically effective amount of the MSC-derived EV-SOX9.

It is to be understood that both the foregoing general description and the following detailed description are exemplary and explanatory only and are not restrictive of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing description and the following detailed description of the invention will be better understood when reading in conjunction with the accompanying drawings. For the purpose of illustrating the present invention, currently preferred embodiments are shown in the drawings.

FIG. 1 shows the chondrogenic progenitor cells in the distinct human MSC were detected using SOX9 and Collagen Type II biomarkers; wherein the MSCs were assessed by flow cytometry for SOX9 and Collagen type II biomarkers indicative of human MSCs on Day 1 and Day 7, as shown by alcian blue staining analyzed the chondrogenic differentiation of pellets with IPFP-MSC, ADSC, and WJ-MSC, which illustrates that the clinical grade of MSC had higher GAG contents consistent with the higher SOX9+Collagen type II⁺-expressing population during chondrogenesis.

FIGS. 2A-2C show the effects of EV-SOX9 in increasing the chondrogenic potential; including FIG. 2(A) shows that the expression of SOX9 mRNA was dramatically detected in the MSC-EV-SOX9 by qPCR; FIG. 2(B) provides images of alcian blue staining analyzed chondrogenic differentiation of pellets with varying ratios of IPFP-MSC from OA patients, showing that EV-SOX9 increased the GAG deposition in IPFP-MSC during chondrogenesis on day 7; FIG. 2(C) shows that EV-SOX9 upregulated the chondrogenic-related genes in the spheroid of IPFP-MSC from the patient suffering from OA.

FIGS. 3A-3D show that the engineered EVs remarkably enhanced cartilage regeneration, including FIG. 3(A) provides images of safranin O staining in the tibial articular cartilage of WT mice after ACLT (scale bars, 100 μm) safranin O staining in mouse joints (n=6) with sham, OA cartilage, and receiving EV/EV-SOX9/IPFP-MSC, indicating that the cartilage thickness and staining intensity were reduced in OA mice by EV/EV-SOX9 administration; FIG. 3(B) shows that OARSI scores indicated articular cartilage damage/repair in all groups; FIG. 3(C) provides images of immunohistological staining (IHC) of collagen II in knee joint sections at 12 weeks after sham operation or ACLT surgery, wherein the IHC staining showed layers of collagen II in distinct groups of articular cartilages; FIG. 3(D) shows the COMP levels with OA disease duration in all study groups (n=6).

FIGS. 4A-4B show that a chondrogenic response of IPFP-MSCs OA patients stimulated by ADSC-EVs expressing SOX9 induction. FIG. 4 (A) provides images of GAG content by day 7 after treating with ADSC-EV-SOX9. FIG. 4 (B) illustrates that ADSC-EV-SOX9 promotes the expression of SOX9, COMP, and ITGB1 genes in IPFP-MSCs from OA patients, as determined by qPCR.

FIG. 5 shows that a higher dosage of naive EVs and EV-SOX9 administered into joints did not cause clinical symptoms, inflammation, or cytotoxic responses.

### DETAILED DESCRIPTION

Unless otherwise defined, all technical and scientific terms used herein have the same meaning as commonly understood by those skilled in the art to which the present invention pertains. It should be understood that the terminology used herein is for the purpose of describing specific embodiments only and is not intended to be limiting.

As used herein, the singular forms “a”, “an” and “the” include plural references unless explicitly indicated otherwise. Thus, for example, reference to “a sample” includes a plurality of such samples and their equivalents known to those skilled in the art.

As used herein, the term “cartilage” includes hyaline cartilage, fibrocartilage, or elastic cartilage, and is not particularly limited. One particular example in the present invention, is knee cartilage.

As used herein, the term “cartilage damage” refers to a disease caused by cartilage defects, injuries, damages or damages caused by cartilage, cartilage tissue and/or joint tissues (synovial membrane, articular capsule, cartilaginous bone, etc.) injured by mechanical stimulation or inflammatory reaction. Such cartilage defect diseases include, but are not limited to, degenerative arthritis, rheumatoid arthritis, fractures, muscle tissue damage, plantar fasciitis, humerus ulcer, calcified myositis, or joint damage caused by fracture nonunion or trauma. In the invention, one particular example is osteoarthritis (OA).

As used herein, the phrase “therapeutically effective amount” refers to an amount sufficient to treat a disease at a reasonable benefit/risk ratio applicable to medical treatment, and an effective dose level may be determined by the species and severity, age, sex, type of disease, duration of treatment, factors including co-administered drugs, and factors well known in other medical disciplines.

As used herein, the term “subject” refers to a human or an animal, including a human or an animal, particularly Canidae such as dogs or wolfs, Felidae such as cats, lions, or tigers, Rodentia such as mouses, rats, hamsters, guinea pigs, and gerbils, Leporidae such as rabbits or hares, Bovidae such as cows or sheep, Camelidae such as camels or alpaca, or Equidae such as horses. In one preferred example of the invention, the subject or patient is a human, In another example of the invention, the subject or patient is a pet animal, such as a dog, cat, hamsters, guinea pigs, rabbits horse, alpaca and etc. In the invention, the subject or patient refers to one with a cartilage damage, or a group in need of a treatment for improving cartilage regeneration or a treatment of a cartilage damage.

As used herein, the term “SOX9” refers to a protein of SRY-box transcription factor 9 (SOX9) in a human or any animal. One example is human SOX9, e.g., SRY-box transcription factor 9 [Homo sapiens (human)]. (Tommerup N, Schempp W, Meinecke P, Pedersen S, Bolund L, Brandt C, et al. (June 1993). “Assignment of an autosomal sex reversal locus (SRA1) and campomelic dysplasia (CMPD1) to 17q24.3-q25.1”. Nature Genetics. 4 (2): 170-4.)

As used herein, the term “extracellular vesicles” or “EVs” refers to lipid bilayer-delimited particles that are naturally released from almost all types of cells but, unlike a cell, cannot replicate. EVs range in diameter from near the size of the smallest physically possible unilamellar liposome (around 20-30 nanometers) to as large as 10 microns or more, although the vast majority of EVs are smaller than 200 nm. EVs can be divided according to size and synthesis route into Exosomes, microvesicles and apoptotic bodies. They carry a cargo of proteins, nucleic acids, lipids, metabolites, and even organelles from the parent cell. Based on current research, most cells are believed to release extracellular vesicles (EVs). Numerous EV subtypes have been proposed, differing definitions based on size, biogenesis pathway, cargo, cellular source, and function.

The present invention provides a novel therapy of a cartilage damage, such as osteoarthritis (OA) using EV-SOX9.

According to the invention, the EV-SOX9 is obtained by encapsulating the SOX9 mRNA or the mRNA of its downstream in extracellular vesicles (EVs). The EV-SOX9 may also be naïve EVs with high expression level of SOX9 gene or its upstream or downstream gene, from different cell sources. In one particular embodiment of the invention, the EV-SOX9 is MSC-derived EV-SOX9, which is obtained by transferring the SOX9 gene, such as SOX9 mRNA into multipotent cells and collecting extracellular vesicles (EVs) from the multipotent cells.

According to the present invention, the MSC-derived EV-SOX9 were used as a specific example. The EVs and method for preparation or delivery are illustrated in You, Y. et al. (“Intradermally delivered mRNA-encapsulating extracellular vesicles for collagen-replacement therapy;” Nat. Biomed. Eng (2023)) and Yang, Z. et al. (“Large-scale generation of functional mRNA-encapsulating exosomes via cellular nanoporation;” Nat Biomed Eng 4, 69-83 (2020)); the entire disclosures of which are hereby incorporated by reference herein.

According to the invention, the pharmaceutical composition may be formulated with a pharmaceutically acceptable carrier by standard or commonly used methods. One example of the pharmaceutically acceptable carrier is hydrogel or water. The EV-SOX9 may be coated with or loaded into lipids, liposomes, or nanoparticles to avoid degradation.

As illustrated in the conventional clinical studies and experimental models, mesenchymal stem cells (MSCs) may contribute to cartilage regeneration and the modulation of the immune microenvironment in osteoarthritis (OA) subjects. multipotent cells may be stem cells such as mesenchymal stem cells (MSCs), fibroblasts such as murine embryonic fibroblasts (MEFs), or 293T cells may be used to obtain a cell preparation. In one particular example of the invention, MSC-derived extracellular vesicles (MSC-EVs) were prepared and found to possess therapeutic properties, and thus it may serve as novel carriers for gene and drug delivery. In the present invention, the SOX9 mRNA is used to engineer EVs to promote chondrogenic induction and cartilage repair in an OA disease animal model, and the master transcription factor, SOX9, is a crucial player in controlling chondrocyte differentiation, and the expression of type II collagen, the major component of the cartilage extracellular matrix, closely parallels that of SOX9.

It is ascertained in the examples that the SOX9 mRNA was significantly detected in the MSC-EV-SOX9 by analyzing the chondrogenic potential from infrapatellar fat-pad-derived MSC (IPFP-MSC), wherein alcian blue staining was used to identify the glycosaminoglycan (GAG) deposition by MSC-EV-SOX9. Furthermore, it was found that GAG synthesis and chondrogenic genes were increased in the IPFP-MSC-treated by EV-SOX9, indicating that EV-SOX9 stimulated the chondrogenesis of IPFP-MSC from the objects with OA. It was observed in the examples that the treatment with MSC-derived EV-SOX9 (which was obtained by transferring SOX9 mRNAs into MSCs and collecting EVs with SOX9) significantly improved cartilage repair in view of the results of safranin O staining and IHC staining of collagen II in the EV-SOX9-treated group as compared with the naïve EV group in the anterior cruciate ligament transection (ACLT) OA mice. The combination of IPFP-MSC and EV-SOX9-treated group had markedly stimulated cartilage repair as compared with the PBS group. Given the above, a remarkable link between EV-SOX9-induced chondrogenesis was concluded and cartilage regeneration was enhanced in ACLT-induced OA. Therefore, the present invention provides an effective therapeutic agent for treating a c, which is EV-SOX9

According to the invention, the EV-SOX9 is formulated with a pharmaceutically acceptable carrier for topical application via injection or a surgery. The pharmaceutical compositions may be formulated in a suitable suspension in one or more carriers, including, but not limited to hydrogel and/or water. In a certain example, the EV-SOX9 may be surgically implanted into the damage of the subject in need thereof.

The present invention is further illustrated by the following examples, which are provided for the purpose of demonstration rather than limitation.

### EXAMPLES

### Materials and Methods

Preparation of EV-SOX9 and the cell preparation for treating OA

To transfer the SOX9 plasmid into IPFP-MSCs and collect extracellular vesicles (EVs), Transwell®-based electroporation was used. IPFP-MSCs were cultured on the surface of a Transwell® insert with 90% confluence overnight, and the SOX9 plasmids (500 ng/uL in DPBS buffer) were pre-loaded in the bottom chamber. The cells were then exposed to an electric field generated by the Xcell System (Bio-Rad), with ten 10 ms pulses applied at either 125 V to facilitate the electroporation through the membrane pores. After electroporation, the donor cells were cultured in a serum-free medium for 24 hours to allow for EV collection. To collect the EVs, the harvested media were centrifuged at 300×g for 5 minutes at 4° C. to remove cells, then transferred to a new tube and centrifuged again at 2000×g for 20 minutes at 4° C. to remove debris. The sample was then filtered using a 0.22 μm filter. The EV-enriched medium was concentrated and diafiltered using tangential flow filtration (TFF) with a 500 kDa MWCO hollow fiber filter (polysulfone, Repligen, Waltham, MA) under sterile conditions to remove soluble proteins, lipid proteins, and small vesicles. Finally, the EV-enriched medium was further concentrated using Amicon® Centrifugal (Merck Millipore) with a 3 kDa MWCO at 4,000×g to achieve the desired volume and EV concentration. The details of the EVs and the preparation as used in the example are provided in US Patent Publication No. 20210054329A1, the entire disclosure of which is hereby incorporated by reference herein.

Research ethics and consent

We recruited the patients with OA (n=13) aged 50-75 years undergoing joint replacement surgery but excluded those with other rheumatic diseases treated with immunosuppressive drugs in the past 3 months. The included patients' IPFP and SC were obtained and immediately processed. All patients signed an informed consent form for the use of their IPFP. The Research Ethics Committee of Far Eastern Memorial Hospital, Taipei, approved the study (111098-F).

### Mice

Wild-type male mice were from a C57BL/6 background and were bred and maintained under specific pathogen-free conditions in the Far Eastern Memorial Hospital animal care unit. All animal work was conducted according to the Association for Assessment and Accreditation of Laboratory Animal Care guidelines. In addition, all animal experiments were approved by the Animal Ethics Committee of the Far Eastern Memorial Hospital (IACUC number: IACUC-2022 (4)-MOST-09).

Isolation of MSCs from IPFPs

The IPFP tissue was washed repeatedly with Dulbecco's phosphate-buffered saline (Gibco, cat. no. 21600) containing 5% penicillin-streptomycin and amphotericin B solution (Gibco, cat. no. 15240). Next, the tissue was minced and subjected to enzymatic digestion with 0.1% type I collagenase (Gibco, cat. no. 17100017) diluted in minimum essential medium α (α-MEM, Gibco, cat. no. 120000) and 1% bovine serum albumin (Sigma, cat. no. A7906) for 18 h at 37° C. After digestion, an equal amount of culture medium containing 10% fetal bovine serum was added to stop collagenase activity. The cell solution was sieved through a 100-μm mesh and centrifuged at 500 g for 5 min. The cell pellets were resuspended in culture medium (supplemented with 10% FBS, 1% penicillin-streptomycin, and amphotericin B) and seeded in a 10-cm culture dish. The medium was changed every 72 h until the cells reached 80%-90% confluence.

### Chondrogenic Differentiation

To obtain a 3D pellet culture, 1×10⁵ MSCs were centrifuged at 1200 rpm for 3 min in polypropylene tubes and cultured for 1 day. To induce chondrogenesis, the pelleted cells were cultured in DMEM-HG (Gibco, cat. no. 121000) containing 1% ITS-Premix (Corning, cat. no. 354352), 1% penicillin streptomycin (Gibco, cat. no. 15240), 1 mM sodium pyruvate (Gibco, cat. no. 11360070), 0.35 mM proline (Sigma-Aldrich, cat. no. P5607), 10 ng/ml TGF-β1 (SinoBio, cat. no. 10804-HNAC), 0.17 mM ascorbic acid 2-phosphate (Sigma-Aldrich, cat. no. A8960), and 100 nM dexamethasone (Sigma-Aldrich, cat. no. D4902). After induction, the chondrogenic differentiation results were evaluated on days 1, 7, 14, and 21.

### ACLT-Induced Knee OA

Surgical ACLT is the most commonly used for generating an animal model of OA. This study used 8-week-old specific-pathogen-free C57BL/6 mice for ACLT. The mice were subjected to ACLT and medial collateral ligament (MCL) transection for establishing an OA mouse model (n=6). First, the knee joints of the hind limbs were incised along the medial edge of the patellar ligament. After the joint capsule was exposed, the ACL and MCL were transected. After 4 weeks, cartilage destruction and inflammation—signs of OA—were observed in the mice.

### Safranin O/Fast Green Staining

After deparaffinization, the sections were stained with hematoxylin for 3 min and differentiated in 1% acid alcohol for 15 s, followed by staining in 0.02% aqueous fast green for 3 min and counterstaining with 0.1% Safranin O for 3 min. Finally, the sections were dehydrated in serial dilutions of ethanol, cleared in xylene, mounted onto glass slides by using neutral gum. Each stained area was observed under a light microscope and photographed using a photomicroscope equipped with a CCD video camera. For Safranin O staining, the results were obtained from the Commission BIOTOOLS laboratory (Taipei, Taiwan).

### Quantitative Real-Time PCR

Human chondrocytes were harvested, and cellular extracts were prepared to assess the expression levels of cytokines and chondrogenic markers using a LightCycler® system (Roche Applied Science). The fold change in gene expression, plotted on the y-axis, was calculated relative to untreated cells for each gene of interest.

### Statistical Analysis

All data are presented as mean±SEM and were analyzed using GraphPad Prism software (Version 6.0). p values were calculated using one-way analysis of variance with a post hoc Bonferroni test with ≥3 replicates for multiple comparisons and using unpaired Student's t test for two groups.

### Results

Mesenchymal stem cells (MSC) drive a more tolerant or anti-inflammatory phenotype of immune cell differentiation and tissue regeneration. Therefore, MSC-derived extracellular vesicles (EVs) have been implicated in having therapeutic effects for osteoarthritis (OA) through immunomodulation and induction of chondrogenesis. However, naïve EVs have been reported to promote the repair and regeneration of damaged cartilage. Still, they had been considered to ineffectively improve cartilage repair as the naïve EVs derived from the MSC source might be from a non-super-donor. We then considered whether potential mRNA targets might contribute to regulating chondrocyte differentiation since the feasibility of engineering EV methods to utilize them as RNA drug delivery carriers. Therefore, for developing more effective MSC EVs, the potential mRNAs loaded in MSC EVs can become effective strategies for cartilage repair. The master transcription factor SOX9 is a key player during chondrocyte differentiation. In addition, the expression of type II collagen, the major component of the cartilage extracellular matrix, closely parallels that of SOX9. Therefore, clarifying the SOX9 mRNAs loaded in MSC EVs (EV-SOX9) treatment underlying OA occurrence and progression is essential to facilitate new therapies for future clinical needs.

For analyzing the chondrogenic potential from infrapatellar fat-pad-derived MSC (IPFP-MSC), we used alcian blue staining to identify the glycosaminoglycan (GAG) deposition. The underlying limitation is that the MSC population is heterogeneous, resulting in difficulty in determining the superior chondrogenic population. Therefore, based on the well-known biomarkers controlling chondrogenesis, we first used the critical biomarkers to identify the chondrogenic precursor lineages from human MSC. Then, the two biomarkers of SOX9 and collagen type II were used to determine the chondrogenic precursor cells by FACS and examine their chondrogenic capacity by GAG assay. The results revealed that the SOX9⁺Collagen type II⁺-expressing was higher in IPFP-MSC from non-OA than OA donors. In addition, the higher GAG content was consistent with, the higher SOX9⁺Collagen type II⁺-expressing population. Moreover, we compared the clinical grade of adipose-derived stem cells (ADSC) and Wharton's jelly MSC (WJ-MSC) cells and showed a greater SOX9⁺Collagen type II⁺-expressing population. These observations suggested that the SOX9⁺Collagen type II⁺-expressing population could be a chondrogenic progenitor with better chondrogenic capacity.

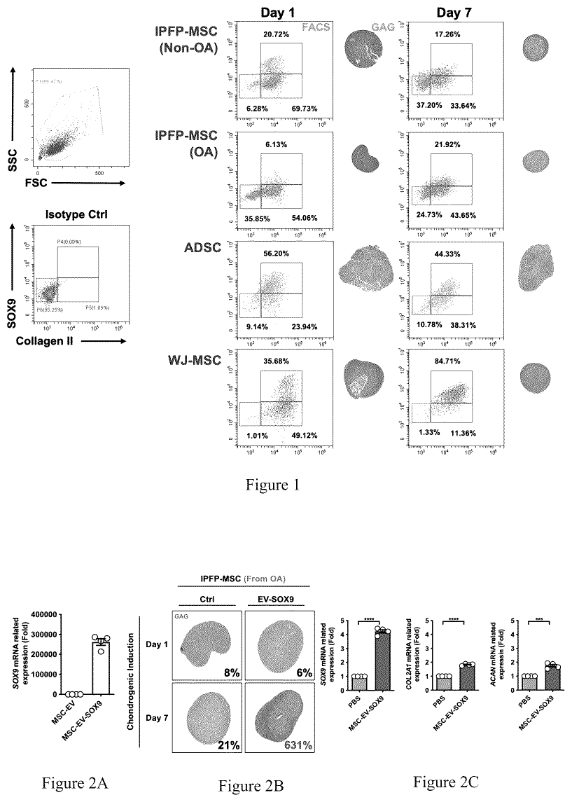

Based on the results of FIG. 1, it was concluded that the SOX9 gene (mRNA) was significantly detected in the MSC-EV-SOX9. Our results showed increased GAG synthesis in the IPFP-MSC-treated with EV-SOX9 compared to the control EV, indicating that EV-SOX9 stimulates the chondrogenesis of IPFP-MSC. To develop more effective MSC EVs, the SOX9 mRNAs loaded in MSC EVs (EV-SOX9) treatment significantly improved cartilage repair by safranin O staining and IHC staining of collagen II and Osteoarthritis Research Society International (OARSI) scores in the EV-SOX9-treated group compared with the naive EV group in the anterior cruciate ligament transection (ACLT) OA mice.

Furthermore, the combination of IPFP-MSC and EV-SOX9-treated group had markedly lower OARSI scores than the EV-SOX9-treated group after ACLT compared with the PBS group.

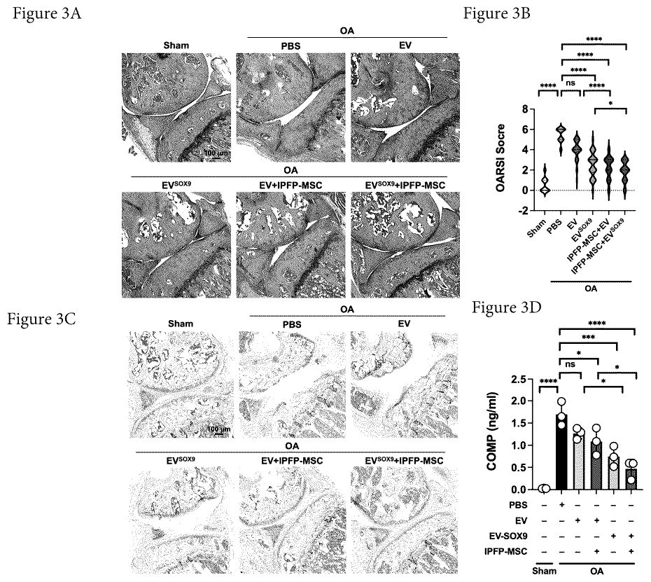

In addition, it was found in FIG. 2 that in PBS-treated OA mice, serum cartilage oligomeric matrix protein (COMP) levels, as a prognostic indicator of future joint damage and as a marker of ongoing joint damage, increased and decreased in PBS-treated OA mice that were administered EV-SOX9 or EV-SOX9 and IPFP-MSCs. Further, as shown in FIG. 3, the engineered EV remarkably enhanced cartilage regeneration, which was supported by the results of safranin O staining in the tibial articular cartilage of WT mice after ACLT. The safranin O staining in mouse joints (n=6) with sham, OA cartilage receiving EV/EV-SOX9/IPFP-MSC was conducted. Based on the reduction of the EV/EV-SOX9 administration, the OARSI scores indicated articular cartilage damage/repair in all groups, the immunohistological staining (IHC) of collagen II in knee joint sections at 12 weeks after sham operation or ACLT surgery (sowing the layers of collagen II in distinct groups of articular cartilages), and the COMP levels with OA disease duration in all study groups as measured (n=6), the EV-SOX9 administration provides an improvement in.

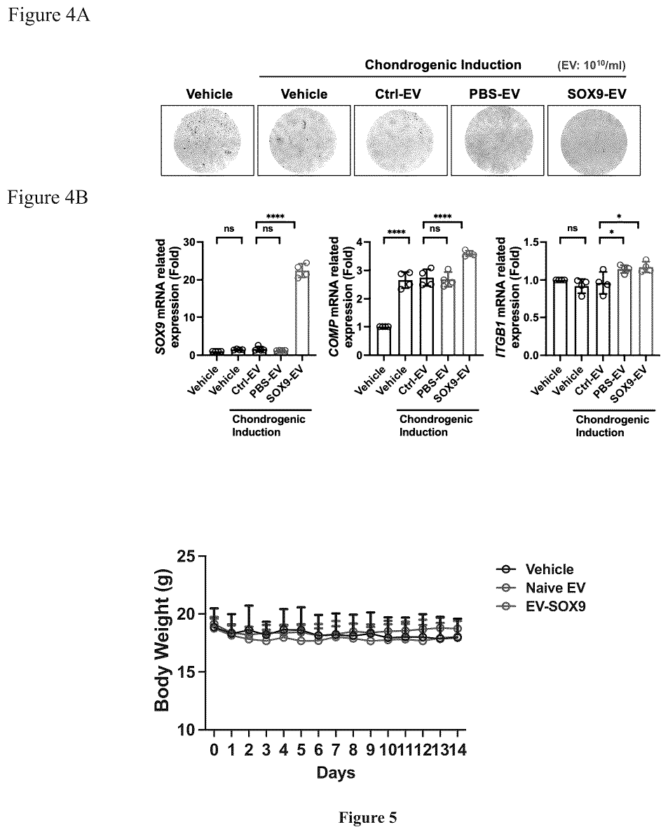

It was found in FIG. 4 that stimulation of IPFP-MSCs derived from OA patients by ADSC-EVs expressing SOX9 can induce a chondrogenic response. The GAG content was obviously increased by introducing SOX9-EV. Further, the mRNA expression level of SOX9, COMP, and ITGB1 was significantly increased by treating SOX9-EV. This example illustrates the extraordinary chondrogenic induction effect of SOX9-EV.

Furthermore, the results showed in FIG. 5 provide that higher dosages of naive EVs and EV-SOX9 did not cause clinical joint symptoms, weight loss, or cytotoxic responses up to day 14. A higher dosage of naive EVs and EV-SOX9 administered into joints did not cause clinical symptoms, inflammation, or cytotoxic responses as performing intra-articular injections of these substances in the joints of mice.

Hyaluronic acid-based hydrogels were found to protect EVs from degradation, extend their bioactivity, and have demonstrated beneficial effects on joint regeneration in OA canine. By combining the MSC-EV-SOX9 biological drug with hydrogels, its observed effects in preclinical settings could provide additional opportunities to improve the management of OA symptoms.

In view of the results, it could be concluded that EV-SOX9 functioned in regulating chondrogenic induction in IPFP-MSC from OA subjects in vitro. Moreover, EV-SOX9 significantly improved cartilage repair in the ACLT-induced OA disease mouse model, which was especially combined with IPFP-MSC administration, canine and human patients. In conclusion, the function of EV-SOX9 was characterized in regulating chondrogenesis compared to the control group.

While this invention has been described in connection with what is presently considered to be practical exemplary embodiments, it is to be understood that the invention is not limited to the disclosed embodiments. On the contrary, it is intended to cover various modifications and equivalent arrangements included within the spirit and scope of the appended claims.

What is claimed is:

1. A method for treating a cartilage damage in a subject, comprising the steps of:
providing an EV-SOX9, which is extracellular vesicle (EV) with SOX9 gene or its upstream or downstream gene;
administrating to the subject a therapeutically effective amount of the EV-SOX9.

2. The method according to claim 1, wherein the downstream gene is Collagen type II (COL2A1) or Aggrecan (ACAN).

3. The method according to claim 1, wherein the EV-SOX9 is obtained by encapsulating the SOX9 mRNA, the mRNA of its upstream or downstream, or an miRNA, which regulates SOX9 in extracellular vesicles (EVs).

4. The method according to claim 1, wherein the EV-SOX9 is naïve EVs with high expression level of SOX9 mRNA or its upstream or downstream gene from different cell sources.

5. The method according to claim 1, wherein the EV-SOX9 is mesenchymal stem cells (MSCs)-derived EV-SOX9, which is obtained by transferring the SOX9 gene or its upstream and downstream gene into mammalian cells and collecting extracellular vesicles (EVs) derived from the mammalian cells.

6. The method according to claim 5, wherein the mammalian cells comprise multipotent cells or fibroblasts.

7. The method according to claim 6, wherein the multipotent cells are mesenchymal stem cells (MSCs).

8. The method according to claim 6, wherein the fibroblasts are murine embryonic fibroblasts (MEFs).

9. The method according to claim 6, wherein the fibroblasts are 293T cells.

10. The method according to claim 1, wherein the EV-SOX9 is administered to the cartilage damage of the subject via an injection or a surgery.

11. The method according to claim 1, wherein the cartilage damage is osteoarthritis (OA).

12. The method according to claim 1, further comprises a combination of a therapy or a therapeutic agent for treating a cartilage damage, such as platelet-rich plasma (PRP), hyaluronic acid (HA), a cell therapy for repair or regeneration of cartilage tissues, and/or a stem cell therapy.

13. The method for treating a cartilage damage in a subject according to claim 1, which comprises the steps of:
providing MSC-derived EV-SOX9, which is obtained by transferring the SOX9 gene or its upstream or downstream gene into mesenchymal stem cells and collecting extracellular vesicles (EVs) as MSC-derived EV-SOX9;
administrating to the subject a therapeutically effective amount of the MSC-derived EV-SOX9.

14. The method according to claim 1, wherein the subject is a human.

15. The method according to claim 1, wherein the subject is an animal.

16. The method according to claim 15, wherein the subject is a pet animal.

17. A pharmaceutical composition for treating a cartilage damage, comprising a therapeutically effective amount of EV-SOX9 as set forth in claim 1, and a pharmaceutically acceptable carrier.

18. The pharmaceutical composition according to claim 17, wherein the EV-SOX9 is obtained by encapsulating the SOX9 mRNA, the mRNA of its upstream or downstream gene, or an miRNA, which regulates SOX9 in extracellular vesicles (EVs).

19. The pharmaceutical composition according to claim 17, wherein the EV-SOX9 is naïve EVs with high expression level of SOX9 mRNA or its upstream or downstream gene from different cell sources.

20. The pharmaceutical composition according to claim 17, wherein the EV-SOX9 is mesenchymal stem cells (MSCs)-derived EV-SOX9, which is obtained by transferring the SOX9 gene or its upstream or downstream gene into mammalian cells and collecting extracellular vesicles (EVs) derived from the mammalian cells.
